SEQUENCE LISTING (6 sequences)

SEQ ID NO: 1 - DNA, 20 bp, Synthetic construct
  source 1..20: mol_type = other DNA
ctgagaggttgagggacgga

SEQ ID NO: 2 - DNA, 20 bp, Synthetic construct
  source 1..20: mol_type = other DNA
ggaagtggcaactgatgagc

SEQ ID NO: 3 - DNA, 20 bp, Synthetic construct
  source 1..20: mol_type = other DNA
gcaaggcacctctgagaact

SEQ ID NO: 4 - DNA, 20 bp, Synthetic construct
  source 1..20: mol_type = other DNA
gggtgagaaaaccacgttgc

SEQ ID NO: 5 - DNA, 20 bp, Synthetic construct
  source 1..20: mol_type = other DNA
cgaaagcgcaagaaatcccg

SEQ ID NO: 6 - DNA, 20 bp, Synthetic construct
  source 1..20: mol_type = other DNA
gctccagggcattagacagc

(19) United States
(12) Patent Application Publication
(10) Pub. No.: US 2023/0372581 A1
(43) Pub. Date: Nov. 23, 2023

(54) NANO-HYPERBARIC WOUND HEALING THERAPEUTIC

(71) Applicants: THE BOARD OF TRUSTEES OF THE UNIVERSITY OF ILLINOIS, Urbana, IL (US); REVIVE BIOTECHNOLOGY, INC., Champaign, IL (US)

(72) Inventors: Joseph Maria Kumar IRUDAYARAJ, Champaign, IL (US); Wen REN, Urbana, IL (US); Michael TSIPURSKY, Champaign, IL (US)

(21) Appl. No.: 18/320,485

(22) Filed: May 19, 2023

Related U.S. Application Data
(60) Provisional application No. 63/344,030, filed on May 19, 2022.

(51) Int. Cl. A61L 26/00

(52) U.S. Cl. CPC A61L 26/008; A61L 26/0014; A61L 26/0004; A61L 26/0095; A61L 26/0023

(57) ABSTRACT

Herein is described an oxygen nanobubbles-embedded hydrogel (ONB-G) with carbopol for oxygenation of wounds to accelerate the wound healing process. We integrate carbopol hydrogel and dextran-based ONBs, to prepare ONB-G that can hold oxygen and release it to accelerate wound healing. Oxygen release tests showed that the proposed ONB-G could maintain oxygen in the hydrogels for up to 34 days. Also, fluorescence studies indicated that the ONB-G could maintain the high oxygen levels for up to 8 weeks. Histological evaluation of tissues with a pig model with incision and punch wounds showed that treatment with ONB-G exhibited improved healing compared with hydrogel without ONBs or treated without gel. Our studies show that dextran-shell ONBs embedded in a gel (ONB-G) has the potential to accelerate wound healing given its oxygen holding capacity and release properties.

### RELATED APPLICATIONS

This application claims priority under 35 U.S.C. § 119(e) to U.S. Provisional Patent Application No. 63/344,030, filed May 19, 2022, which is incorporated herein by reference.

### REFERENCE TO SEQUENCE LISTING

The present application includes a Sequence Listing in electronic format as an xml file titled “500.146US1 SL” which was created on May 3, 2023 and has a size of 6,383 bytes. The contents of xml file 500.146US1_SL are incorporated herein by reference.

### BACKGROUND OF THE INVENTION

Acute or Chronic wounds located on the largest organ, the skin, of humans often result in suffering and in the long term, significant morbidity to patients. It is estimated that there are currently about 10 million people in the United States suffer from diabetic foot ulcers, one of the most common chronic wounds. Meanwhile, acute wounds are also quite common with injuries and in postoperative setting. Development of treatment options for chronic wounds will reduce morbidity which can result in loss of a limb and even life. Expedited recovery from surgical wounds can reduce patient suffering and improve surgical outcomes. It has been suggested that oxygen would accelerate the complex wound healing process, which encompasses multiple biological events such as cell proliferation and protein production (Adv Wound Care (New Rochelle) 2012, 1(6), 225). To accelerate wound healing, one of the most common treatments recommended is oxygenation in a hyperbaric chamber where pressurized oxygen in the controlled environment can be used to hyperoxygenate tissues. However, hyperbaric chambers are not commonplace and are not easily accessible due to logistics of insurance approvals and need for travel on daily basis to a facility, risk of exposure to high pressure oxygen, large size of the chambers, and imposes an unnecessary economic burden on the patient and the system. Portable devices for oxygen delivery such as Topical Wound Oxygen (TWO2®) Therapy and OxyGeni® exists but are potentially risky due to the risk of explosion from oxygen use/generation and poor wound penetration and thus have not had good adoption by the physician community Therefore, a need exists in the development of facile methods for oxygenation of wounds to accelerate healing.

Hydrogels are ideal materials for wound dressing and provide an optimal microenvironment for the wound and have been used along with antibiotics or other compounds for wound care. Hydrogels are also a conducive matrix for oxygen delivery or generation at the wound surface to improve the healing process. Current products for oxygen provision to the wounds are generally grouped into the following categories: i) hydrogel where oxygen is generated from H₂O₂ or other substrates in a catalyzed reaction; or ii) hydrogel with bioactive species which would generate oxygen.

The amount of oxygen in the hydrogel based on dissolved oxygen is determined by the solubility of oxygen in the solvent. Although in some of the gel materials, chemicals such as perfluorocarbons have been used to improve the solubility of oxygen, delivery of oxygen for a prolonged period of time is limited. Oxygen-generating reactions such as catalyzed H₂O₂ hydrolysis could provide sufficient oxygen in a hydrogel-based wound dressing. However, to guarantee the reaction only occurs when the hydrogel is applied to the wounds, the substrates and catalysts should not interact prior to the application of the dressing to the wound. For the hydrogels containing catalysts such as MnO₂, H₂O₂ needs to be stored separately and added in an incremental manner, making the application of the dressing more complex. For the hydrogels encapsulating H₂O₂, the hydrolysis of H₂O₂ prior to application and the potential reaction between H₂O₂ and the hydrogel component could influence the utility of hydrogel materials in healthcare. Oxygen-providing hydrogel has been produced by integrating hydrogel and bioactive species such as microalgae or bacteria which could produce oxygen through photosynthesis. However, the other products beyond oxygen generated during the bioactivity of these species also pose a concern due to the possible harm to patients' health, necessitating further investigation.

Oxygen nanobubbles (ONBs) are oxygen-encapsulating shells primarily based on materials such as polymers or lipids (Int. J. Pharm. 2009, 381 (2), 160). Compared with dissolved oxygen, ONBs could store oxygen for an extended period of time when present in a suitable buffer. In our own prior work, we have shown that ONBs can be used as ultrasound contrast agents and in cancer therapy. More recently we have shown that ONBs could be used to treat central retinal artery occlusion, an ischemic condition of the retina. Evaluation of ONBs and their characterization in several retinal cells also showed excellent promise of tissue preservation due to extended oxygenation.

There exists a need for improved devices, like for a wound dressing which is easy to self-apply and that can oxygenate chronic and acute wounds to promote healing.

### SUMMARY

The integration of ONBs in a carbopol-based hydrogel enabled the development of new materials with excellent oxygen-holding capacity with extended-release characteristics to successfully treat oxygen deficit indications in acute as well as chronic wounds. The effect of carbopol concentration on the oxygen release capacity and rheological features of the ONB-G were investigated along with the sterility of ONB-G. HDFa cell-based studies were conducted to evaluate the viability, proliferation, and revival of cells in hypoxia. Scratch assay and mRNA expression studies indicated the potential benefit for wound closure.

Accordingly, this disclosure provides a hydrogel composition comprising:
a) a crosslinked polyacrylic acid gel;
b) oxygen nanobubbles comprising polysaccharide colloidal shells that encapsulate oxygen;
   wherein:

the shells comprise dextran, trehalose, lecithin, palmitic acid, and tocopherol; and

the shells have an average outer diameter of about 450 nm or less; and
c) an electrolyte;
   wherein the oxygen nanobubbles are distributed throughout the hydrogel composition.

This disclosure also provides a bandage for healing of a wound configured for the application of the hydrogel composition described above to the wound.

Additionally, this disclosure provides a method for healing a wound, comprising:
applying the hydrogel composition described above to a subject having a wound in need of healing; and
allowing the oxygen nanobubbles distributed throughout the hydrogel to disintegrate with time, wherein the oxygen encapsulated by the polysaccharide colloidal shells are released into the crosslinked polyacrylic acid gel to provide a continuous supply of oxygen to the wound for 5 days or more;
thereby enhancing healing of the wound.

This technology provides for the use of the compositions described herein for use in treating and healing wounds in a subject.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. Oxygen concentration curve from ONB-G, hydrogel prepared with oxygen-saturated water as positive control, and without hydrogel as the negative control (A). Calculated oxygen release for 12 hours from ONB-G stored at 4° C. (B). Fluorescence at 590 nm from Ru(dpp)₃²⁺ in ONB-G and hydrogel without ONBs stored at serial times (C).

FIG. 2A-F. Oxygen release from ONB-G prepared with various concentrations of carbopol 940 (A). Rheological characterization of ONB-G by amplitude sweep (B) and frequency sweep (C). Frequency test of hydrogel without ONBs (D) as control. Frequency sweep characterization of ONB s-containing gel prepared with 1.5% (E) and 2% (F) carbopol 940.

FIG. 3A-B. Bacterial studies for assessing sterility of the hydrogels. Sterility experiments follow U.S. Pharmacopoeia Sterility <71> protocol (A). Minimum inhibitory concentration against Staphylococcus aureas and various amounts of ONB-G (B).

FIG. 4A-B. HDFa cell viability studies. HDFa cells cultured in normoxic conditions and treated with ONB-G at 2 mg or 5 mg concentration (A). HDFa cell viability in hypoxia treated with either 2 mg or 5 mg ONB-G (B). Data shown as mean±standard deviation. n=8. “n.s.” indicates no significant difference between groups. ** p<0.01, **** p<0.0001.

FIG. 5A-B. HDFa scratch assay. HDFa cells cultured in hypoxic conditions before treatment (i) and after incubated in hypoxia with no treatment (ii), 2 mg ONB-G (iii), 2 mg hydrogel without ONBs (iv), 5 mg ONB-G (v), or 5 mg hydrogel without ONBs (vi). The Red dashed line indicates HDFa cell borders (A, Scale bar=100 μm). HDFa cell viability in hypoxia treated with either 2 mg or 5 mg ONB-G, or 2 mg or 5 mg of hydrogels without ONBs (B). Data shown as mean±standard deviation. n=4. * p<0.05, *** p<0.001.

FIG. 6A-C. RT-PCR analysis of hypoxic genes. HDFa cells were cultured in either normoxic or hypoxic (3% 02) conditions for 6 hours. Evaluation of mRNA expression of HIF-1α. The ONB-G has a significant effect on the HIF-1α expression at both the 2 and 5 mg dose levels (A). Evaluation of mRNA expression of VEGF-A (B). Evaluation of mRNA expression of PAI-1 (C). † significantly different from the NT Normoxia group. significantly different from the NT Hypoxia group. * p<0.05.

FIG. 7A-C. Histological sections of 8 mm punch wounds stained with H&E. Sections include punch wounds treated with Tegaderm (A), hydrogel without ONBs (B), or ONB-G (C). The dotted boxes in column 1 correspond to the magnified images in columns 2, 3, and 4. Closed arrows indicate re-epithelialization. Open arrows indicate immature epithelialization. Other highlighted sections are the blood clot (BC), adipose tissue (AT), granulation tissue (GT), and dermal collagen (DC). The scale bar in the original image is 2.5 mm. Scale bar in the magnified image is 250 μm.

FIG. 8A-C. Histological sections of incision wounds stained with H&E. Sections include incisions treated with Tegaderm (A), hydrogel without ONBs (B), or ONB-G (C). The magnified images of the dotted boxes in column 1 is provided in columns 2 and 3. Highlighted areas include dermal collagen (DC), adipose tissue (AT), and blood clot (BC). The scale bar in the original image is 2.5 mm. Scale bar in the magnified images is 250 μm.

### DETAILED DESCRIPTION

Herein, we report on the development of ONB s-embedded hydrogel (ONB-G) based on carbopol for wound healing applications. Carbopol (polyacrylic acid) is a polymer with high molecular weight and has been used in hydrogel preparation for drug delivery on patients' skin (Drug Dev. Ind. Pharm. 2020, 46 (5), 706). Furthermore, carbopol has been shown to have enhanced gelling properties (gels can be formed in 10 min), thus reducing the loss of oxygen during hydrogel preparation. The gel formation occurs at room temperature, making it a low-temperature process. The oxygen release and storage capability of the proposed ONB-G were first evaluated. We show that the hydrogel could continuously release oxygen for up to 46 hours. Compared with the hydrogel prepared with oxygen-saturated water, ONB-G provides a significantly increased amount of oxygen. Meanwhile, the oxygen release capability of the ONB-G remained at a similar level after 32 days of storage, exhibiting excellent stability of oxygen storage in the hydrogel. We further investigated the effect of carbopol concentration on oxygen release from the corresponding hydrogel. It is noted that although the rheological properties of the hydrogel vary with the level of carbopol, the oxygen release features of these hydrogels are similar. We have also shown that ONB-G is sterile, i.e., prevents bacterial growth and was nontoxic to adult human dermal fibroblasts (HDFa). Further experiments show that ONB-G can significantly increase wound closure in a scratch assay when applied to HDFa cells in hypoxia. mRNA expression studies show that the ONB-G significantly reduced PAI-1, which is upregulated in hypoxia and contributes to impaired wound closure. In vivo acute wound studies in a pig model demonstrated significant closure of the punch biopsy wounds and positively remodeled incision wounds as seen from histology staining. Compared with the wounds treated with hydrogel without ONBs or Tegaderm only, the histological results of the tissues from wounds treated with ONB-G exhibited improved healing benefiting from the oxygenated hydrogel dressing with ONBs.

Additional information and data supporting the invention can be found in the following publications by the inventors: Transl. Vis. Sci. Technol. 2023; 12(2):16, and its Supporting Information; and WO2022/115323, which publications are incorporated herein by reference in their entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering”, “introducing”, are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

A “solvent” as described herein can include water or an organic solvent. Examples of organic solvents include hydrocarbons such as hexane, and heptane; ethers such as diethyl ether, tetrahydrofuran, and dibutyl ether; ketones such as acetone and 2-butanone; esters such as ethyl acetate and butyl acetate; alcohols such as methanol, ethanol, and tert-butanol; and aprotic polar solvents such as N,N-dimethylformamide (DMF), N,N-dimethylacetamide (DMA), and dimethyl sulfoxide (DMSO). Solvents may be used alone or two or more of them may be mixed for use to provide a “solvent system”.

The term “molecular weight” for the polymers disclosed herein refers to the average number molecular weight (Mₙ). The corresponding weight average molecular weight (M_{w}) can be determined from other disclosed parameters by methods (e.g., by calculation) known to the skilled artisan.

### EMBODIMENTS OF THE TECHNOLOGY

This disclosure provides a hydrogel composition comprising:
a) a crosslinked polyacrylic acid (PAA) gel;
b) oxygen nanobubbles (ONBs) comprising polysaccharide colloidal shells that encapsulate oxygen;
   wherein:

the shells comprise dextran, trehalose, lecithin, palmitic acid, and tocopherol; and

the shells have an average outer diameter of about 450 nm or less; and
c) an electrolyte;
   wherein the oxygen nanobubbles are distributed throughout the hydrogel composition.

In some embodiments, the crosslinked PAA is carbopol 940. In some embodiments, the hydrogel composition comprises the medicament. In some other embodiments, the medicament is an active agent that promotes wound healing. In other embodiments, the medicament is an anti-bacterial agent.

References to oxygen (O₂) herein, can mean dissolved oxygen or oxygen gas.

In various embodiments, the crosslinked polyacrylic acid gel does not comprise unencapsulated oxygen. In various embodiments, the shells comprise about 2×10⁻² wt % to about ×10⁻² wt % dextran. In various embodiments, dextran has an average molecular weight of about 100 kDa to about 1000 kDa, or preferably about 300 kDa, about 400 kDa, about 500 kDa, about 600 kDa, about 700 kDa, about 800 kDa, about 900 kDa, or about 1000 kDa. In various embodiments, the shells comprise about 0.2×10⁻² wt % to about 5×10⁻² wt % trehalose. In various embodiments, the shells comprise about 0.2×10⁻² wt % to about 5×10⁻² wt % lecithin. In various embodiments, the lecithin is a soy lecithin. In various embodiments, the shells comprise about 2×10⁻³ wt % to about 10×10⁻³ wt % palmitic acid. In various embodiments, the shells comprise about 3×10⁻⁴ wt % to about 20×10⁻⁴ wt % tocopherol. In various embodiments, tocopherol is alpha-tocopherol polyethylene glycol succinate (TPGS).

In some embodiments, the hydrogel composition comprises at least 1 wt % of the crosslinked polyacrylic acid (PAA) gel. In some embodiments, the hydrogel composition comprises crosslinked PAA in the following amounts in wt. %: 1%, 2%, 3%, 4%, 5%, 6%, 7%, 8%, 9%, about 10%, about 15%, about 20%, about 25%, about 30%, about 35%, about 40%, about 45%, about 50%, about 60%, about 70%, about 80%, or about 90%.

In various embodiments, the electrolyte comprises a mineral salt. In various embodiments, the hydrogel composition comprises about 2×10⁻⁴ wt % to about 20×10⁻⁴ wt % of the mineral salt. In various embodiments, the mineral salt is potassium chloride.

In some embodiments, the oxygen nanobubbles have a zeta potential or an average zeta potential of about −65 mV to about −50 mV. In some embodiments, the average outer diameter of the oxygen nanobubbles is about 165 nm to about 450 nm. In some embodiments, to increase the amount of oxygen stored in the hydrogel composition, the composition comprises about 1 wt % PAA (carbopol), the polysaccharide colloidal shells comprise about 6.73×10⁻² wt % dextran, about 1.20×10⁻² wt % trehalose, about 1.71×10⁻² wt % lecithin, about 4.27×10⁻³ wt % palmitic acid, about 8.38×10⁻⁴ wt % tocopherol (TPGS), and the electrolyte comprises about 6.73×10⁻⁴ wt % potassium chloride.

Also, this disclosure provides a bandage for healing a wound configured for application of the hydrogel composition described herein to the wound.

In various embodiments, the bandage is configured to provide a continuous supply of oxygen to a wound for 5 days or more. In various embodiments, the bandage is an adhesive patch, or compress.

Additionally, this disclosure provides a method for healing a wound, comprising: applying the hydrogel composition described herein to a subject having a wound in need of healing; and allowing the oxygen nanobubbles distributed throughout the hydrogel to disintegrate with time, wherein the oxygen encapsulated by the polysaccharide colloidal shells are released into the crosslinked polyacrylic acid gel to provide a continuous supply of oxygen to the wound for 1 day, 2 days, 3 days, 4 days, about 5 days, about 7 days, or more; thereby enhancing the healing of the wound. The ONB-G dressing can be reapplied to the effected skin at different intervals to provide continuous oxygenation.

In some embodiments, warming the hydrogel could increase the rate of disintegration of the oxygen nanobubble and/or increase the release of oxygen supplied to the wound. In some embodiments, the subject suffers from a wound resulting from peripheral artery disease (PAD), diabetic foot, diabetic ulcers, bed sores, post operative wounds, skin burns, skin grafts, cosmetic applications or procedures, or any other topical application for the purpose of administering oxygen to the injured skin. Other applications include post operative wounds, skin burns, skin grafts, cosmetic applications such as healing after dermabrasion procedure and skin cuts.

### Results and Discussion.

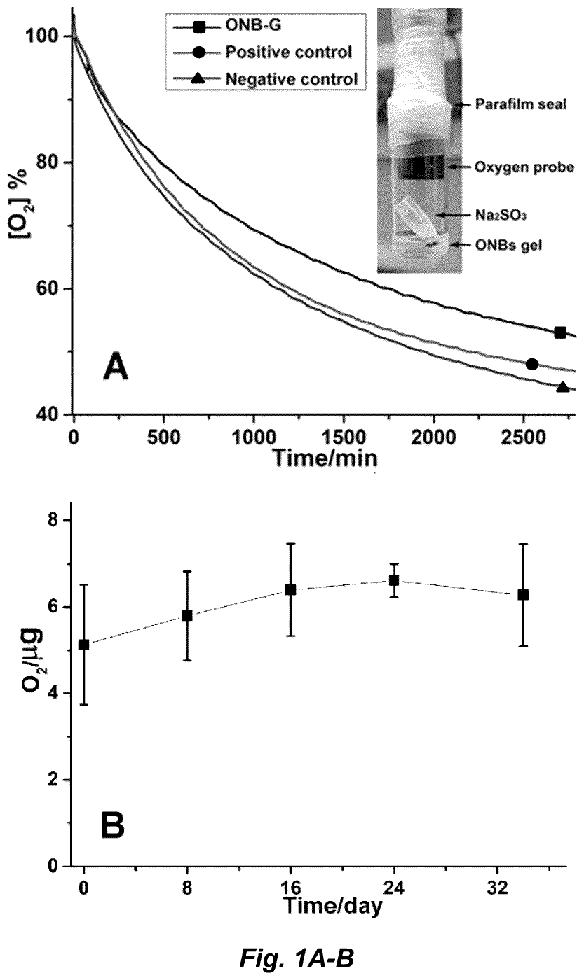

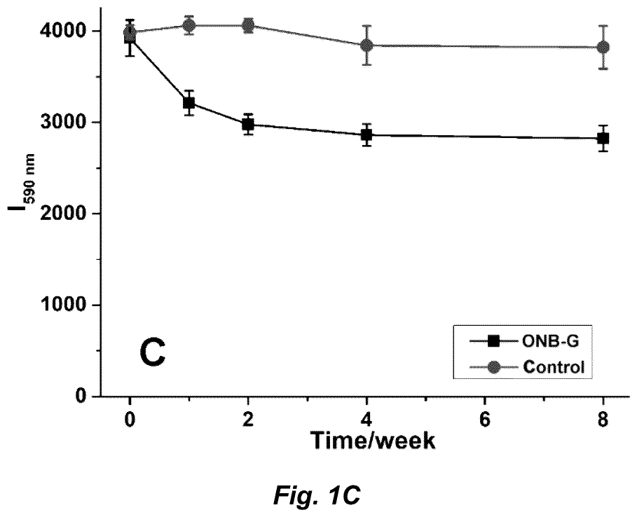

To investigate oxygen release from ONB-G, tests were conducted per the experimental setup shown as the inset in FIG. 1A. The solution with sodium sulfite and cobalt (II) chloride in the centrifuge tube would continuously consume the oxygen in the sealed test system. The change in oxygen level in the test system due to oxygen consumption is depicted as the negative control in FIG. 1A. An oxygen concentration curve shows a consistent decrease in oxygen as demonstrated from the consumption of oxygen in the system with sodium sulfite. When the positive control of the hydrogel prepared with oxygen-saturated water was injected into the test system, a slightly higher level of oxygen was observed compared with the negative control. It is proposed that the increase in the oxygen level is due to the oxygen-saturated water utilized in the positive control. When the ONB-G was injected into the test system, an oxygen concentration curve with a much higher oxygen level was obtained. It can be noted from the test system that the ONB-G sustained a higher oxygen level than the positive control hydrogel and negative control hydrogel. The difference in oxygen level confirmed that the ONB-G has the capacity to deliver oxygen due to the embedded ONBs.

Based on the difference in oxygen concentration curves from ONB-G and the negative control, a calculation was performed to evaluate the amount of oxygen released from ONB-G in 12 hours:

w_{oxygen}=(C_{0h NC}−C_{12h NC})×V−(C_{0h ONBs}−C_{12h ONBs})×(V−1),

where w_{oxygen} is the weight of oxygen released from the ONB-G hydrogel, C_{0h NC} and C_{12 h NC} are the oxygen concentration at the initial and at 12 hours in relation to the negative control, C_{0h ONBs} and C_{12h ONBs} denote the initial and 12-hr oxygen concentration in the ONB-G, and V is the volume of the space in the test setup. The calculation provides an approximate indication of the amount of oxygen from ONB-G. FIG. 1B shows the oxygen released in 12 hours from ONB-G upon storage. A slight increase was noted in the average amount of oxygen released from freshly prepared ONB-G compared to the stored ONB-G hydrogel, meanwhile no observable decrease in the oxygen release capacity of ONB-G was noted and was similar to the 34-day storage, indicating the long-term oxygen storage potential of the ONB-G.

To further confirm the oxygen storage capability in the ONB-G hydrogel, Ru(dpp)₃²⁺, a fluorophore sensitive to oxygen, was used to track the change in oxygen level in the ONB-G. It is known that the fluorescence from Ru(dpp)₃²⁺ will decrease in the presence of oxygen, making it suitable for monitoring oxygen in the hydrogel. The fluorescent intensity at 590 nm was used to evaluate the change in fluorescence of Ru(dpp)₃²⁺ in the hydrogel and the results were shown in FIG. 1C. It can be seen that there is a distinct decrease in the fluorescent intensity from the ONB-G after 1 week; in contrast, the fluorescent intensity in the control, hydrogel without ONBs showed no significant change. The difference between the ONB-G and hydrogel without ONBs indicated the oxygen released from the ONBs embedded in the hydrogel. Furthermore, it can be noted that the decrease in the fluorescence from Ru(dpp)₃²⁺ continued until week 4, indicating the long-term potential for oxygen release from ONB-G.

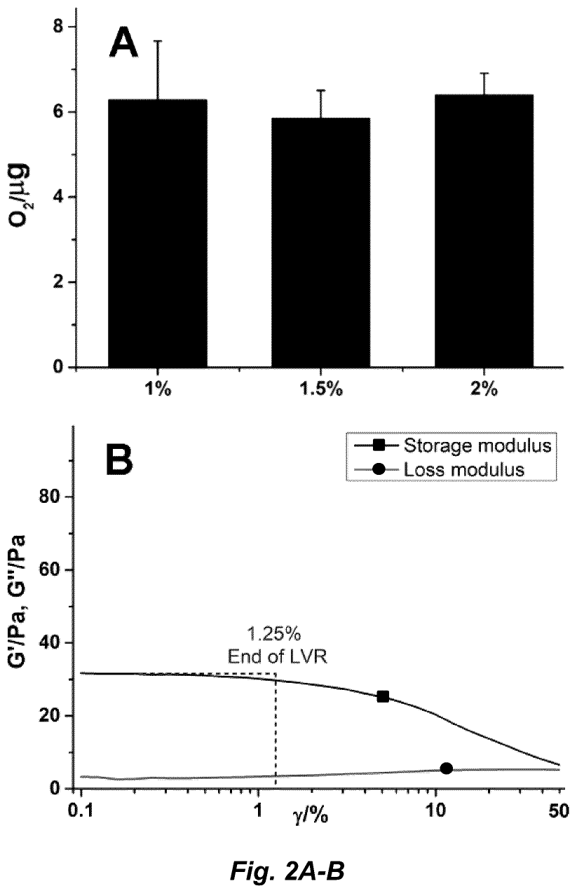

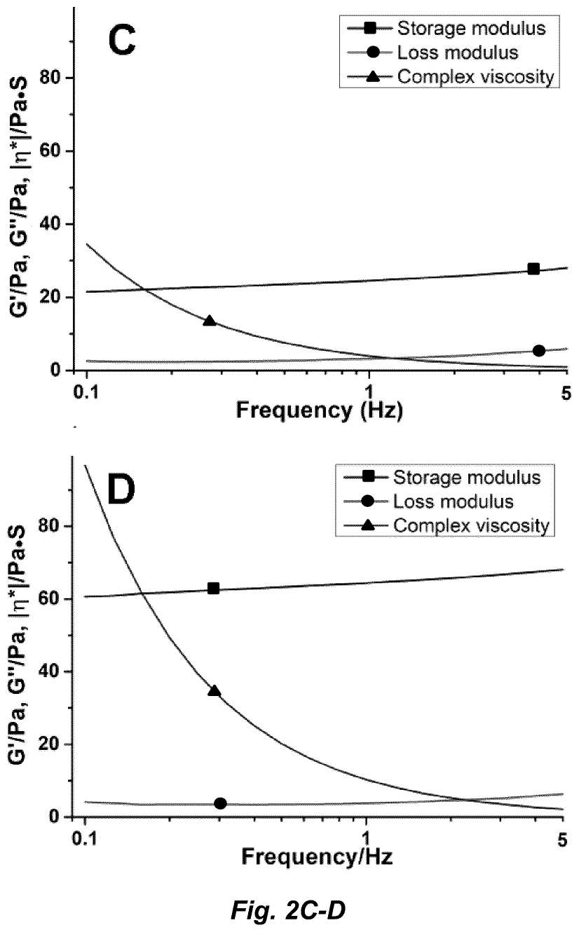

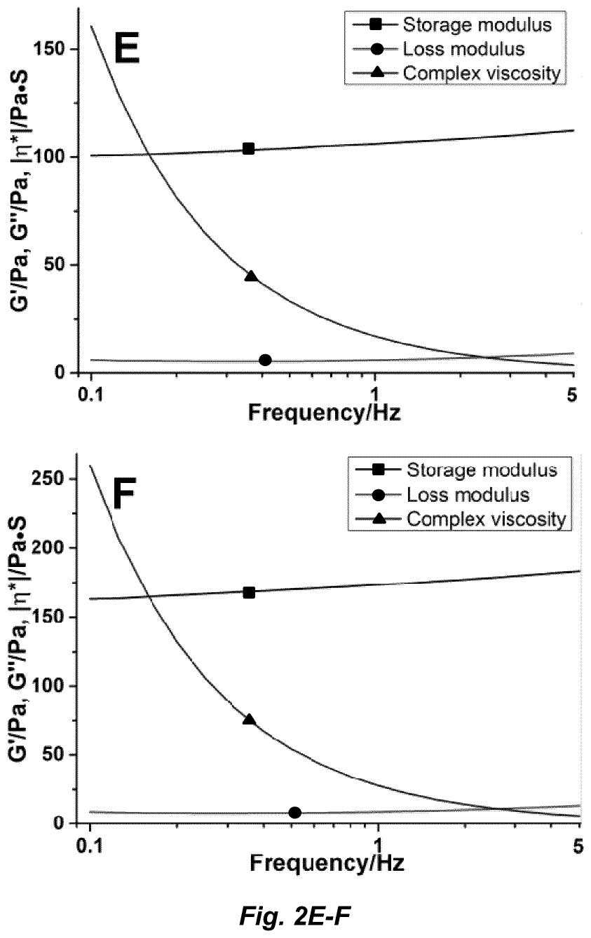

The effect of carbopol concentration in the hydrogel on the oxygen release capability was also evaluated. ONB-G was prepared with carbopol at 1%, 1.5% and 2%. The oxygen released from the ONB-G was tested and the results are shown in FIG. 2A. The release profiles show that the oxygen content released from these ONB-G are similar, indicating that the effect of carbopol concentration on the oxygen release property in the hydrogel is minimal.

The rheological properties of the ONB-G gel were characterized and the results are shown in FIG. 2B-2F. The amplitude sweep test at the frequency of 1 Hz (FIG. 2B) indicated that the linear viscoelastic region of the ONB-G hydrogel is below a strain level of 1.25%. Meanwhile, at a strain level of less than 50%, the storage modulus (G′) is always higher than the loss modulus (G″), indicating a solid-like viscoelastic feature of the ONB-G. FIG. 2C shows the results from frequency sweep tests with ONB-G evaluated at a strain level of 1%. It can be noted that a larger G′ than G″ in the range from 0.1 Hz to 5 Hz, highlighting the dominant elastic features of the ONB-G gel. The complex viscosity (|η*|) decreased with an increase in frequency, indicating a shear-thinning characteristic of the ONB-G. The hydrogel without ONBs was also tested and its frequency sweep results were shown in FIG. 2D. The significant difference in storage modulus, loss modulus and complex viscosity between ONBG gel and hydrogel without ONBs indicated that the presence of ONBs in the hydrogel would influence the viscoelastic behavior of the hydrogel. Meanwhile, as in FIG. 2E and FIG. 2F, the test results from hydrogels fabricated with 1.5% and 2% carbopol 940 showed that the rheological properties of the hydrogel depend on the concentration of carbopol 940 in the ONB-G product.

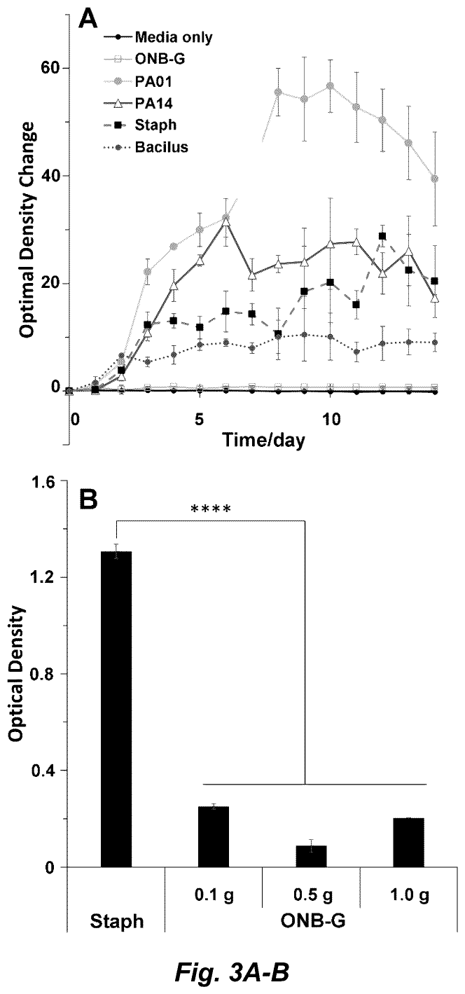

It is essential that the ONB-G hydrogel is sterile and not conducive for bacteria growth. The sterility of the hydrogel was evaluated following the U.S. Pharmacopeoia <71> Sterility protocol. From FIG. 3A, the optical density of the ONB-G hydrogel and media-only samples did not increase from the baseline for 14 days, indicating that there is no bacterial growth. In contrast, samples of Bacillus subtilis, Staphylococcus aureus, Pseudomonas aeruginosa 14 (PA14), and Pseudomonas aeruginosa 01 (PA01) exhibited a sharp increase in optical density after 48 hours. Additionally, the minimum inhibitory concentration was tested against Staphylococcus aureus with varying amount of the hydrogel. As shown in FIG. 3B, the hydrogel weights of 0.1 g, 0.5 g, and 1.0 g exhibited significantly lower bacterial growth after 24 hours of incubation. This shows that the hydrogel is synthesized aseptically and can inhibit bacteria growth.

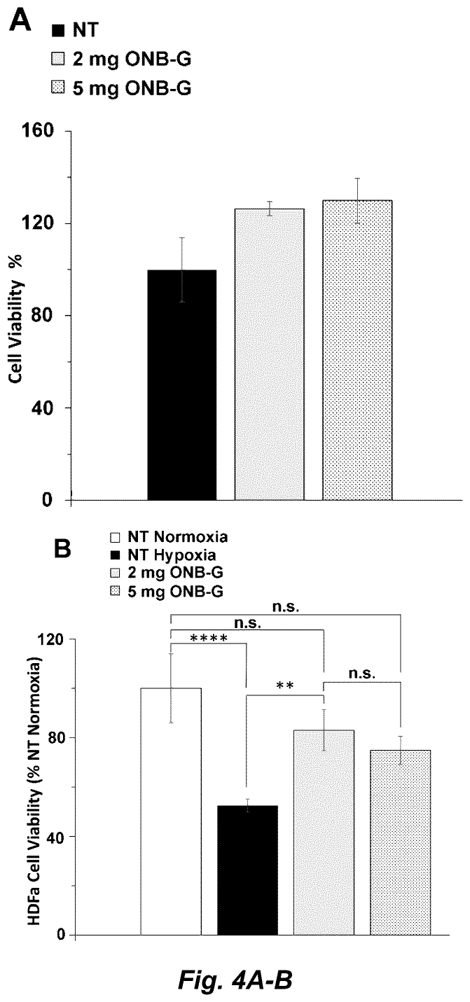

Another essential requirement is that the hydrogel does not damage the dermis. Therefore, viability tests with HDFa skin cells were evaluated. The skin cells were treated with 2 mg or 5 mg hydrogel, and at normoxia (20% 02) and no cell death was noted as indicated by ˜100% viability in each tested group. In hypoxia (3% 02), with the untreated cells under normoxic conditions as the reference with 100% viability, the untreated cells had a viability of 56.6±7.03%, compared to the 2 mg dose which had 85.5±8.31% and the 5 mg dose had 74.9±5.72% viability (FIG. 4). The presence of the ONB-G hydrogels significantly increased cell viability when cultured in a hypoxic environment. Additionally, there was no significant difference between either 2 mg or 5 mg ONB-G hydrogels in hypoxia compared to that of HDFa cells cultured in normoxic conditions. Therefore, the ONB-G hydrogel promoted a conducive environment for HDFa cell growth that is similar to that in normoxic conditions. Here, the ONB-G hydrogel did not cause cell death but promoted cell survival in hypoxic environments.

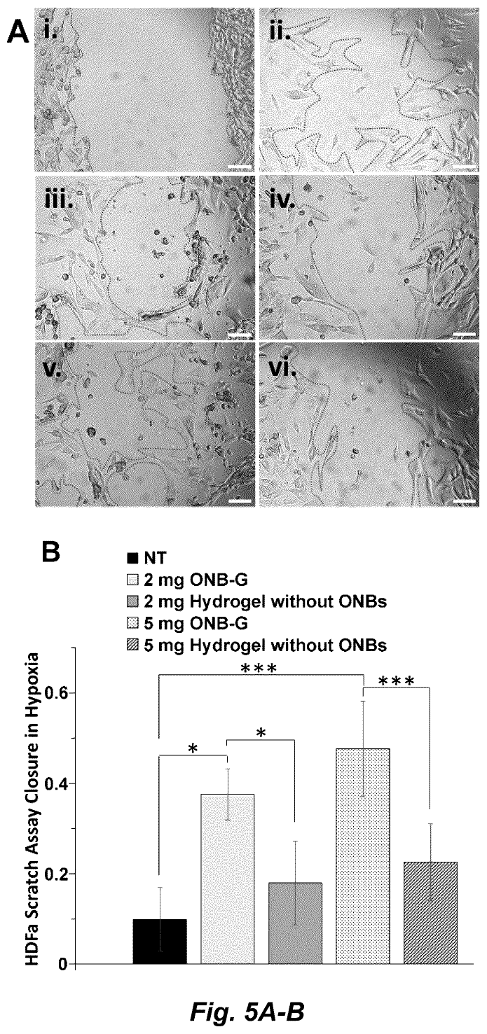

To test the efficacy of the ONB-G hydrogel, a scratch assay was performed in a hypoxic environment. After 24 hours of incubation in hypoxia with either 2 mg or 5 mg of ONB-G, 2 mg or 5 mg of hydrogel without ONBs, or no treatment, there is a significant difference between treated and untreated cells. Compared to the initial scratch distance shown in FIG. 5A (i), the ONB-G hydrogels (FIG. 5A (iii) and FIG. 5A (v)) had substantial growth to close the simulated wound. The hydrogel without ONBs (FIG. 5A (iv) and FIG. 5A (vi)) did not provide an adequate environment for wound closure. However, increased wound closure was noted in the hydrogels without ONBs than in the no-treatment group. This could indicate that properties intrinsic to the carbopol hydrogel can promote cell growth and wound closure.

To quantify the scratch assay, the distance between the wound edges was measured via the NIH ImageJ software. FIG. 5B shows that both the 2 mg and 5 mg ONB-G hydrogels had significantly more scratch closure than the no-treatment group. Additionally, the presence of ONBs had a significant effect on wound closure. The hydrogel without ONBs at both doses had more scratch closure than the no-treatment group, but it was not significant, indicating that carbopol 940 has innate properties that could promote wound healing. Our results are consistent with migration studies of primary cells from skin biopsies38. Under hypoxic conditions, the keratinocytes had significantly less migration than those cultured under normoxic conditions. With our ONB-G, the cells were able to increase their cell migration properties even in hypoxia.

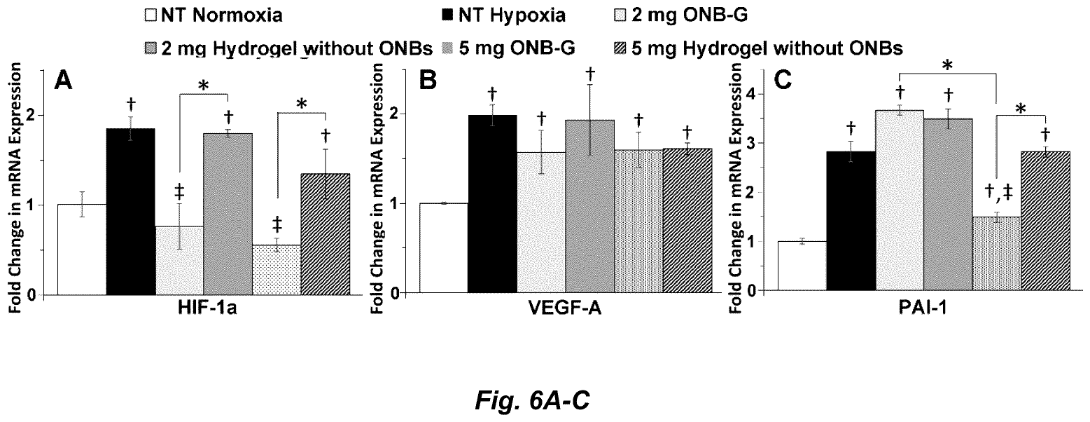

Under hypoxic conditions, various genes were affected. Genes that are upregulated in hypoxic or wounded environments are HIF-1α, VEGF-a and PAI-1. It has been shown that HIF-1α and VEGF-a are upregulated in response to hypoxia to promote vasculogenesis, erythropoiesis, and other pathways to increase oxygen delivery to the hypoxic area. PAI-1 is upregulated in hypoxia and is an enzyme known to inhibit the cleavage plasminogen to form plasmin, which results in the inhibition of fibrinolysis in wounds and blood clots. In our study, we found that HIF-1α and PAI-1 were expressed at a significantly lower rate compared to the NT Normoxia group. Both the 2 mg and 5 mg ONB-G hydrogels provided sufficient oxygen to lower the HIF-1α levels significantly from the NT hypoxia group. Additionally, it is shown that the ONBs present in the hydrogel contribute to the downregulation of HIF-1α (FIG. 6A). Additionally, the 5 mg ONB-G hydrogel significantly lowered the expression of PAI-1 mRNA compared to the NT hypoxia group, but the 2 mg ONB-G hydrogel did not have this effect (FIG. 6C). The additional oxygen stored in the 5 mg ONB-G hydrogel allowed the cells to lower the PAI-1 expression, but it was still significantly higher than the cells in the NT Normoxia group. Lastly, VEGF-A was not significantly altered in hypoxia after treatment with the hydrogels. There is a slight decrease in VEGF-A expression in the 2 mg ONB-G compared to the 2 mg hydrogel without ONB s, but it was not significant (FIG. 6B).

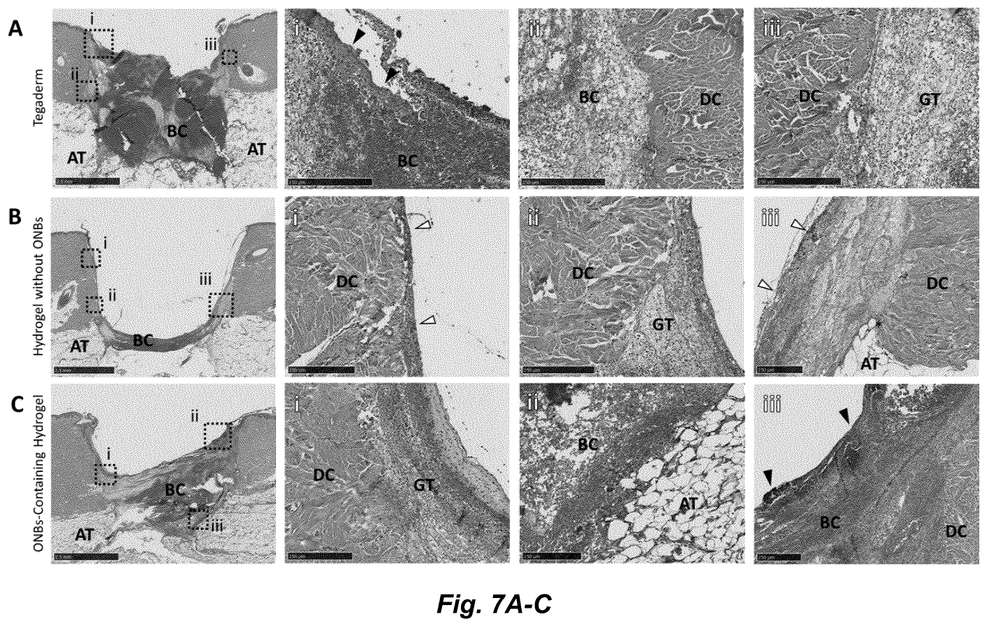

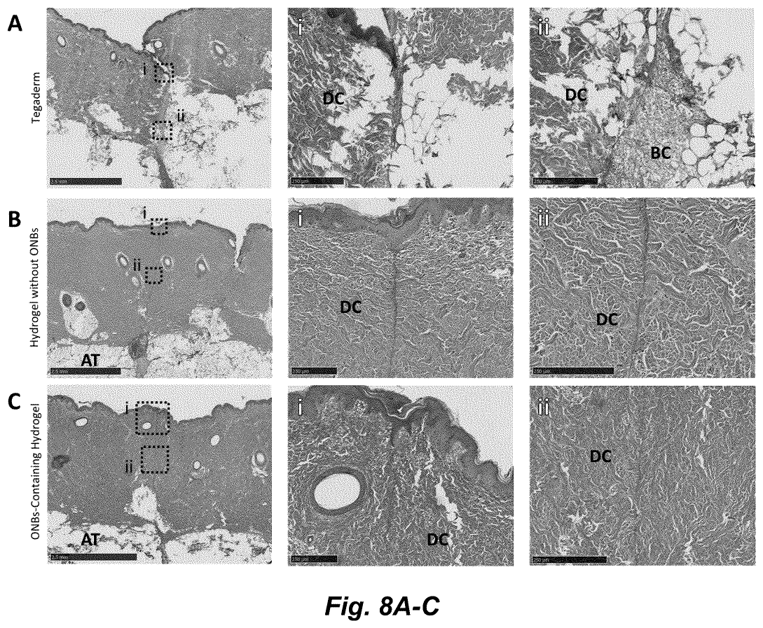

The porcine in vivo model was treated with both the ONB-G and the hydrogel without ONBs. Tegaderm, which is a commercially available wound dressing film, was used as a negative control. After 3 days of treatment, tissues from punch wounds and incision wounds were collected and processed. Histological sections showed that after 3 days of wound healing, varying amounts of granulation tissue were present underneath the blood clot. As shown in FIG. 7, the punch wound treated with ONB-G hydrogel (FIG. 7A) had a lower wound depth excluding the blood clot compared to the Tegaderm and hydrogel without ONBs (FIGS. 7B and 7C). Additionally, the wound treated with ONB-G hydrogel shows signs of re-epithelialization across the wound bed, shown as filled in arrows (FIG. 7C (iii)). Although the Tegaderm and hydrogel without ONBs have re-epithelialization, it is either immature (FIG. 7B (i)) or did not span the entire wound bed (FIG. 7A (i)). In the incision wound, the Tegaderm treated incision did not close to the top of the wound (FIG. 8A (i)); in contrast, a clear closure can be observed on the top of the wounds treated with hydrogel without ONBs (FIG. 8B (i)) or ONB-G (FIG. 8C (i)). Further, in the hydrogel without ONBs, there is blood in the incision wound, whereas in the incision wound treated with ONB-G, there is no blood and the wound fused more clearly. The difference between the wounds treated with carbopol-based hydrogel, containing ONBs or without, and with Tegaderm showed that the dressing with carbopol-based hydrogel would benefit the wound healing process. Furthermore, it should be noted that the wounds treated with ONB-G healed even faster, especially at the deep section of the wound, than the wounds treated with hydrogel without ONBs, demonstrating that the oxygen delivered by the ONBs in the hydrogel would further accelerate the healing procedure.

Conclusion. Herein we propose oxygen nanobubbles-embedded carbopol-based hydrogel as a wound dressing material that could hold and deliver oxygen to the sites of wound to accelerate healing. We show that ONB-G could slowly release oxygen to relieve wounds of hypoxia to maintain normoxic conditions. The proposed ONB-G could store oxygen for up to 34 days. It is noted that an increase in carbopol concentration in the ONB-G did not influence oxygen release capacity of the hydrogel, though the rheological features of the ONB-G were altered with carbopol concentration. Sterility tests show that the fabricated ONB-G was sterile and HDFa cell-based investigations with ONB-G exhibited excellent potential in reviving cells in hypoxia. Experiments with a pig model with incision and punch wounds validated the wound healing characteristics of ONB-G. Histological studies confirm that the carbopol-based hydrogel with embedded ONBs could significantly accelerate wound healing as showing in our acute wound studies. The proposed approach shows promise and could be further developed to treat both acute and chronic wounds as a substitute for hyperbaric oxygen chamber as well as accelerate the healing of acute surgical wounds.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Synthesis of ONBs and Hydrogels

Materials and agents. Sodium sulfite, cobalt(II) chloride hexahydrate, D-(+)-trehalose dehydrate, potassium chloride, dextran sulfate sodium, D-α-tocopherol polyethylene glycol 1000 succinate (TPGS), palmitic acid, and dextrose were purchased from Sigma-Aldrich (MO, US). Water used is Molecular Biology Grade water purchased from Mediatech. Inc. (VA, US). Tris(4,7-diphenyl-1,10-phenanthroline)ruthenium(II) (Ru(dpp)₃²⁺) dichloride and sodium chloride were obtained from VWR International, LLC. Lecithin (DS-Soya PC80-C) is a gift from Solus Advanced Materials Co., Ltd. (Gyeonggi-do, S. Korea. Carbopol 940 was provided by Lubrizol Life Science (OH, US). Ethanol was obtained from Decon Labs Inc (PA, US). Casein Peptone was purchased from Remel Products (Waltham, MA). Peptone S (Soy Peptone) was purchased from BioWorld (Dublin, OH). Dibasic potassium phosphate was purchased from Mallinckrodt Chemicals (Dublin, Ireland). All chemicals were used as received without further purification. Glassware for experiments was cleaned with the detergent and rinsed with DI water and autoclaved.

Preparation of ONBs. The preparation of ONBs was performed based on our previous work with minor changes (Translational Vision Science & Technology 2023, 12 (2), 16). Briefly, oxygen was blown into 10 ml DI water at a pressure of 30 psi. The DI water was sonicated at 50 W with a 30 s sonication-on and 40 s sonication-off cycle. Under sonication, 0.2 ml of 0.045% potassium chloride aqua solution, 1 ml of 0.23% PC80 aqua solution, 0.4 ml of 0.028% TPGS aqua solution, 1 ml of 0.9% dextran aqua solution, 0.3 ml of 0.19% palmitic acid ethanol solution and 0.4 ml of 0.4% trehalose aqua solution was added every two sonication cycles. The obtained solution was sonicated for 4 more cycles and filtered with a 0.22 μm filter. The filtered solution with ONBs was used to prepare ONB-G.

Preparation of ONB-G. Preparation of ONB-G was initiated by the addition of a calculated amount of carbopol 940 directly into the ONBs solution. The obtained mixture was then strongly vortexed for 20 min at room temperature for the complete dissolution of carbopol 940. Gas bubbles generated during the vortex in the obtained mixture were eliminated by centrifugation at 8000 rpm for 30 s. Then ONB-G without gas bubble was thus fabricated. The hydrogel dressing obtained was stored at 4° C. in a sealed glass vial with an aluminum cap and rubber stopper for successive characterization.

### Example 2. Characterization of Hydrogel Compositions

Test of oxygen release. To investigate the oxygen release from the ONB-G, a test system was set up as illustrated in the inset of FIG. 1A. An oxygen meter probe was sealed in a glass vial with a centrifuge tube containing 0.3 ml solution with 0.267 g/ml sodium sulfite and 99 μg/ml cobalt (II) chloride hexahydrate. Then 1 ml of hydrogel sample was injected into the vial and then sealed again with parafilm. A Thermo Scientific™ Orion™ Versa Star Pro™ DO Benchtop Meter with optical dissolved oxygen probes (Ottawa, Ontario, Canada) was used to record the change in oxygen level in the vial at 1-minute intervals.

Fluorescence characterization. To investigate the change in oxygen level in the hydrogel, Ru(dpp)₃²⁺ was added to the carbopol 940 and ONBs solution mixture before vortexing. The concentration of Ru(dpp)₃²⁺ was adjusted to 66 μg/ml in the gel and the fluorescence at 590 nm was recorded in 100 μl hydrogel sample volume in a 96-well plate with a BioTek Synergy HT plate reader.

Rheology characterization. Rheological characterization was performed with an ARES-G2 oscillatory rheometer. ONB-G with 1%, 1.5% and 2% (w/v) of carbopol 940 was evaluated for rheological properties, while hydrogel without ONBs was used as the control.

Bacterial Minimum inhibitory content. Soybean-Casein media was made following the U.S. Pharmacopeia Sterility <71> recipe. Briefly, 1.7 w/v % Casein Peptone, 0.3 w/v % Peptone S (Soy Peptone), 0.5 w/v % Sodium Chloride 0.5, 0.25 w/v % Dibasic Potassium Phosphate 0.25, and 0.23 w/v % Dextrose were dissolved in 1 L of purified water. The pH was adjusted to 7.3±0.2 using 1M NaOH and sterilized via autoclave.

Hydrogels were prepared via an aseptic technique as described herein the day before experiments. The following day, 0.1, 0.5, or 1.0 g (n=3) of hydrogel was placed in separate round bottom tubes and fresh soybean-casein media was added to reach a volume of 3 mL. Staphylococcus aureas at 0.5×108 CFU/mL was evaluated as a positive control, and media only (no inoculation) was used as the negative control. The samples were inoculated with Staphylococcus aureas at 0.5×108 CFU/mL unless otherwise specified, incubated for 48 hours at 37° C., and agitated at 300 RPM. The optical density was read using the Eppendorf's BioPhotometer with 1 mL of each sample and a 1×PBS solution was used as blank.

Evaluation of Hydrogel Sterility. Soybean-Casein media was prepared as above, and the sterility test followed the U.S Pharmacopeia Sterility <71> guidelines. Hydrogel samples were made the day before and added to sterile 50 mL tubes at n=3. Media was used as a negative control. For positive controls, media was inoculated with the following: Bacillus subtilis, Staphylococcus aureus, Pseudomonas aeruginosa 14 (PA14), and Pseudomonas aeruginosa 01 (PA01). The samples were incubated at room temperature for 14 days. The optical density was read each day with 1 mL of the sample in a clean cuvette with 1×PBS as a blank. The sample was returned to the respective 50 mL tubes and incubated at room temperature.

Cell Culture. Primary Human Dermal Fibroblasts (HDFa, PCS-201-012, ATCC) cells were cultured in Fibroblast Basal Medium (PCS-201-013, ATCC) supplemented with the Fibroblast Growth Kit-Low Serum (PCS-201-041, ATCC), 1×Antimycotic-Antibiotic (15240096, Thermo Fisher) at 37° C., 5% CO₂.

Cell Viability. Normoxic Conditions: Cells were seeded at 5,000 cells/well in 96-well plates and incubated overnight with eight replicates. The next day, fresh media was exchanged and the cells were treated with either 2 mg or 5 mg of ONB-G, or 2 mg, or 5 mg hydrogel without ONBs for 24 hours. The following day, MTT Assay was performed to evaluate cell viability. Hypoxic Conditions: HDFa cells were seeded in a similar manner as above in 96-well plates. After overnight attachment, the media was removed and replaced with fresh media and the cells were then treated with either 2 mg or 5 mg ONB-G and placed in a humidified hypoxic chamber with 3% O₂, 5% CO₂, and 92% N₂ at 37° C. for 24 hours. The next day, the media was exchanged for fresh media and MTT assay was performed.

Scratch Assay. The day before the experiment, cells were seeded at 5×10⁴ cells/well in Ibidi culture two well inserts (80209, Gräfelfing, Germany) with n=2 and cultured overnight to ensure cell attachment. The next morning, the cell culture inserts were removed and images were taken at 4× magnification in bright field. The cells were then treated with either ONB-G at 2 mg or 5 mg, or hydrogel alone at 2 mg or 5 mg, and placed in a humidified hypoxic chamber (3% O₂, 5% CO₂, and 92% N₂) at 37° C. for 24 hours. The following day, the media was replaced with fresh media, and images were taken again at 4× magnification. For quantitative analysis, 4 images per well of different areas of the scratch assay were taken. ImageJ was utilized to measure the distance between the two sides of the scratch at the initial and final time points.

The percent of scratch closure was determined per the equation:

$$Scratch Closure = \frac{Initial Distance - Final Distance}{Initial Distance}$$

Hypoxic Gene Expression. The day before the experiment, cells were seeded at 0.6×10⁶ cells/well at n=2 in a 6-well plate. The following day, the cells were given fresh media and treated with 2 mg or 5 mg of the ONB-G or no treatment. The cells were placed in a humidified hypoxic chamber for 6 hours in the same manner as before. Cells were then washed with 1×PBS, trypsinized, pelleted, and washed again with 1×PBS. The total RNA was extracted with ThermoFisher's RNA Purification kit (K0731, Waltham, MA) following manufacturer's instructions. cDNA was synthesized with Applied Biosystem's High-Capacity cDNA Reverse Transcription kit (4368814, Waltham, MA) following manufacturer's instructions (Table 1). RT-PCR analysis was conducted using Applied Biosystem's PowerUp SYBR Green Master Mix (A25742, Waltham, MA) for PCR following manufacturer's instructions with n=3 and 2 ng of cDNA per well.

TABLE 1
The primers used to amplify the targets are:

| Primer | Forward | Reverse |
|---|---|---|
| HIF-1α | CTG AGA GGT TGA | GGA AGT GGC AAC |
| | GGG ACG GA | TGA TGA GC |
| | (SEQ ID NO: 1) | (SEQ ID NO: 2) |
| PAI-1 | GCA AGG CAC CTC | GGG TGA GAA AAC |
| | TGA GAA CT | CAC GTT GC |
| | (SEQ ID NO: 3) | (SEQ ID NO: 4) |
| VEGF-A | CGA AAG CGC AAG | GCT CCA GGG CAT |
| | AAA TCC CG | TAG ACA GC |
| | (SEQ ID NO: 5) | (SEQ ID NO: 6) |

Animal husbandry and Wound Treatment. In vivo experiments with a pig model were conducted per the protocol (IACUC Protocol #: 22133) approved by the University of Illinois Urbana-Champaign. An intramuscular injection of ceftiofur (5 mg/kg) was given to the animal 5 to 7 days before surgery. Additionally, the animal was made to fast for 12 hours before the surgery. On the day of surgery, an intramuscular anesthetic of a telazol-atropine-rompun-ketamine cocktail was administered, and the surgical area was shaved and cleaned.

A sterile 8 mm biopsy punch was utilized to create identical wounds for n=3 per treatment with 1 cm spacing. The wound was then pressure held with gauze to stop excessive bleeding. Next, 3 incisions approximately 2 cm long were made through the dermis and fascia. The incisions were closed with non-dissolvable silk sutures.

The wounds were treated with ONB-G, or hydrogel without ONBs, and covered with a 6×6 cm PVC sheet to prevent oxygen release away from the wound. Wounds with no treatment were used as a negative control. All wounds were covered with Nexcare's Tegaderm transparent dressing (H1626, St. Paul, MN). Benamine S was given intramuscularly at 2.3 mg/kg prior to surgery and prophylactically every 12 hours. On day 3 after surgery, the animal was sacrificed, tissues excised and fixed in 4% neutral buffered saline.

Statistical Analysis. Statistical analysis was conducted with the R Studio statistical software. For MTT analysis, eight replicas were evaluated. Bacterial minimum inhibitory concentration, RT-PCR, sterility, and wound analysis were evaluated with 3 replicates. The scratch assay had two replicates, with four measurements taken per image. A one-way ANOVA followed by a post hoc Tukey test was used to determine the statistical difference between groups. Results were considered significant if the resulting p-value was less than 0.05.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A hydrogel composition comprising:
a) a crosslinked polyacrylic acid gel;
b) oxygen nanobubbles comprising polysaccharide colloidal shells that encapsulate oxygen;
wherein the shells comprise dextran, trehalose, lecithin, palmitic acid, and tocopherol; and the shells have an average outer diameter of about 450 nm or less; and
c) an electrolyte;
wherein the oxygen nanobubbles are distributed throughout the hydrogel composition.

2. The hydrogel composition of claim 1 wherein the crosslinked polyacrylic acid gel does not comprise unencapsulated oxygen.

3. The hydrogel composition of claim 1 wherein the shells comprise about 2×10⁻² wt % to about 20×10⁻² wt % dextran.

4. The hydrogel composition of claim 3 wherein dextran has an average molecular weight of about 100 kDa to about 1000 kDa.

5. The hydrogel composition of claim 1 wherein the shells comprise about 0.2×10⁻² wt % to about 5×10⁻² wt % trehalose.

6. The hydrogel composition of claim 1 wherein the shells comprise about 0.2×10⁻² wt % to about 5×10⁻² wt % lecithin.

7. The hydrogel composition of claim 6 wherein the lecithin is a soy lecithin.

8. The hydrogel composition of claim 1 wherein the shells comprise about 2×10⁻³ wt % to about 10×10⁻³ wt % palmitic acid.

9. The hydrogel composition of claim 1 wherein the shells comprise about 3×10⁻⁴ wt % to about 20×10⁻⁴ wt % tocopherol.

10. The hydrogel composition of claim 9 wherein tocopherol is alpha-tocopherol polyethylene glycol succinate (TPGS).

11. The hydrogel composition of claim 1 wherein the composition comprises at least 1 wt % of the crosslinked polyacrylic acid gel.

12. The hydrogel composition of claim 1 wherein the electrolyte comprises a mineral salt, and the composition comprises about 2×10′ wt % to about 20×10⁻⁴ wt % of the mineral salt.

13. The hydrogel composition of claim 12 wherein the mineral salt is potassium chloride.

14. The hydrogel composition of claim 1 wherein the oxygen nanobubbles have a zeta potential of about −65 mV to about −50 mV, and the average outer diameter of the oxygen nanobubbles is about 165 nm to about 450 nm.

15. The hydrogel composition of claim 1 wherein the polysaccharide colloidal shells comprise about 6.7×10⁻² wt % dextran, about 1.2×10⁻² wt % trehalose, about 1.7×10⁻² wt % lecithin, about 4.3×10⁻³ wt % palmitic acid, about 8.4×10⁻⁴ wt % tocopherol, and the electrolyte comprises about 6.7×10⁻⁴ wt % potassium chloride.

16. A bandage for healing a wound configured for application of the hydrogel composition of claim 1 to the wound.

17. The bandage of claim 16 wherein the bandage is configured to provide a continuous supply of oxygen to the wound for 5 days or more.

18. The bandage of claim 16 wherein the bandage is a compress, or an adhesive patch.

19. A method for healing a wound, comprising:
applying the hydrogel composition of claim 1 to a subject having a wound in need of healing; and
allowing the oxygen nanobubbles distributed throughout the hydrogel to disintegrate with time, wherein the oxygen encapsulated by the polysaccharide colloidal shells are released into the crosslinked polyacrylic acid gel to provide a continuous supply of oxygen to the wound for 5 days or more;
thereby enhancing healing of the wound.

20. The method of claim 16 wherein the subject suffers from a wound resulting from peripheral artery disease (PAD), diabetic ulcers, bed sores, post operative wounds, skin burns, skin grafts, or cosmetic applications.
